(19) United States
(12) Patent Application Publication
(10) Pub. No.: US 2007/0155660 A1
(43) Pub. Date: Jul. 5, 2007

(54) Inhibition of voluntary ethanol consumption with selective melanocortin 4-receptor agonists

(76) Inventors: Donald J. Marsh, Hillsborough, NJ (US); Todd E. Thiele, Chapel Hill, NC (US)

Correspondence Address: MERCK AND CO., INC, P O BOX 2000, RAHWAY, NJ, 07065-0907, US

(21) Appl. No.: 10/581,166

(22) PCT Filed: Dec. 6, 2004
(86) PCT No.: PCT/US04/40616

§ 371 Date: May 31, 2006

Related U.S. Application Data
(60) Provisional application No. 60/528,326, filed on Dec. 10, 2003.

(51) Int. Cl. A61K 38/12

(52) U.S. Cl. 514/9

(57) ABSTRACT

The present invention relates to methods of inhibiting or reducing voluntary alcohol consumption in a subject comprising administering a selective melanocortin 4 receptor agonist to said subject. The present invention further relates to methods of treating or preventing alcoholism, alcohol abuse, and alcohol related disorders in a subject comprising administering a selective melanocortin 4 receptor agonist to said subject. The present invention further provides for pharmaceutical compositions and medicaments useful in carrying out these methods.

### BACKGROUND OF THE INVENTION

Alcohol abuse is one of the most significant problems in modem society. Nearly 14 million people in the United States, approximately 1 in every 13 adults, abuse alcohol or are alcoholics (U.S. Dept. of Human Services, 2001 National Household Survey on Drug Abuse: Volume 1 (BKD461, SMA 02-3758)). According to the National Institutes of Health, each year alcohol abuse accounts for 45% of all car crash fatalities (over 20,000 individuals) and is involved in approximately 44% of all short-stay hospital visits. An additional 26,000 individuals die from alcohol-associated chronic liver disease and cirrhosis of the liver (NCHS, National Vital Statistics Report Vol. 50, No.5, 2000). The Justice Department reported that alcohol was involved in nearly 40% of all violent crimes in 1998. The resulting economic cost of alcohol abuse to the United States is estimated to be nearly $150 billion per year.

The causes of alcoholism are not fully known. Genetics may play a role; a family history of alcoholism makes it more likely for a person to develop alcoholism if that person chooses to drink. Certain environmental risk factors may also influence whether a person with a genetic risk for alcoholism ever develops the disease.

Alcohol problems may be classified into two categories, alcoholism or alcohol dependence, and alcohol abuse. Alcoholism is a dependence on alcohol and is characterized by abnormal alcohol seeking behavior that leads to impaired control over drinking. Alcohol abuse is characterized by drinking too much or too often, without being an alcoholic. Alcohol misuse has also been found to predispose the subject to osteoporosis, slow bone healing, impaired wound healing, inhibited osteoblastic function and diminished immune defenses. Alcohol intoxication increases the risk of further accidents, and decreases the pain inhibition that would make a normal patient more careful. Alcohol dependence also leads to altered cognitive and emotional functions, such as impaired judgment, feelings of incompetency, low self-esteem, despair in relationships, feelings of failure, and depression.

Several medications are currently used to treat alcoholism. Disulfiram (Antabuse®) and Naltrexone (Trexan®) are the only FDA approved products that are currently available for adjunctive use in the treatment of alcohol abuse. Disulfiram works by blocking the intermediary metabolism of alcohol in the body to produce a build up of acetaldehyde, which in turn produces markedly adverse behavioral and physiological effects. Patient compliance in taking the drug is poor due to these side effects (see T W Rall, in: Goodman and Gilman's The Pharmacological Basis of Therapeutics, A G Gilman et al, 8th Edition, Chap 17, pp 378-379). Naltrexone is a well-known narcotic antagonist and is thought to work by blocking activation of the endogenous opiate reward system, which may be activated by alcohol consumption. In practice, naltrexone is only moderately effective because it is relatively short acting and patients require co-treatment with behavioral therapy for the drug to have any effect (J R Volpicelli et al, Arch Gen Psychiatry, 1992, 49:876-880). Benzodiazepines (Valium®, Librium®) are also sometimes useful during the first days after patients stop drinking to help them safely withdraw from alcohol; however these medications can not be used for longer periods because they are highly addictive. As a result, there is a continuing need to develop new compounds that are useful for the treatment of alcoholism and alcohol abuse in mammals.

Recent studies support a role for melanocortin signaling in behavioral and neurochemical actions of ethanol. The melanocortin (MC) system is composed of peptides that are cleaved from the polypeptide precursor, proopiomelanocortin (POMC). These peptides include adrenocorticotropic hormone (ACTH), (α-melanocyte stimulating hormone (α-MSH), β-MSH, and γ-MSH. Brain melanocortin peptides are produced primarily by neurons within the hypothalamic arcuate nucleus, the nucleus of the solitary tract, and the medulla. Genetic and pharmacological evidence reveals that melanocortin signaling is involved with grooming behavior, antipyretic and anti-inflammatory responses, learning, reproductive function, and regulation of appetite and energy homeostasis.

A recent report found significant differences in brain melanocortin 3 and melanocortin 4 receptor levels between rats selectively bred for high ethanol consumption (AA) and low ethanol consumption (ANA). AA rats selectively bred for high ethanol drinking have lower levels of melanocortin 3 receptor in the shell of the nucleus accumbens when compared with controls, but have high levels of melanocortin 3 receptor (MC3R) and melanocortin 4 receptor (MC4R) in various regions of the hypothalamus (Lindblom et al., Pharn Biochem Behav 72:491-496, 2002). It has been shown that central infusion of the non-selective melanocortin agonist, MTII, significantly reduces voluntary ethanol drinking and prevents ethanol-induced changes in endogenous opioid peptide levels in the substantia nigra and VTA of AA rats with an established ethanol intake (Ploj et al., Brain Res Bull, 59:97-104, 2002). It was suggested that melanocortin signaling may regulate ethanol drinking by modulating endogenous opioid activity within mesolimbic dopamine pathways (Ploj et al., Brain Res Bull, 59:97-104, 2002). However, a recent report indicated that a MC4R-selective antagonist (HS014) has no effect on ethanol drinking by AA rats (Ploj et al., Brain Res Bull, 59:97-104, 2002).

It is unclear which melanocortin receptor(s) are important for modulating MTII-induced reductions of ethanol consumption (Ploj et al., Brain Res Bull, 59:97-104, 2002). MTII is a non-selective melanocortin agonist that binds, with varying affinity, to all centrally expressed melanocortin receptors (MC1R, MC2R, MC3R, MC4R and MC5R), but has the greatest affinity for MC3R and MC4R (Haskell-Luevano et al., J. Med. Chem. 40:1738-1748, 1997; Schioth et al., Peptides, 18:1009-1013, 1997). It is also possible that melanocortin 1 receptor (MC1R) and/or melanocortin 5 receptor (MC5R) are involved, as MTII binds to both of these receptors (Haskell-Luevano et al., J. Med. Chem. 40:1738-1748, 1997; Schioth et al., Peptides, 18:1009-1013, 1997). MC1R is expressed specifically in periaqueductal gray (PG) region of the brain (Xia et al., NeuroReport, 6:2193-2196, 1995) while MC5R is found in several brain regions, including the NAc (Griffon et al., Biochem Biophys Res Com, 200:1007-1014, 1994).

The selective MC4R agonists of the present invention are beneficial over non-selective melanocortin agonists since selective MC4R agonists do not exhibit the side effects associated with non-selective MC4R agonists, such as MC1mediated pigment changes and worsening of acne associated with MC5R agonists.

It has now been found that selective melanocortin 4 receptor agonists are useful to inhibit alcohol consumption. The present invention shows that melanocortin 4 receptor is the primary melanocortin receptor involved with regulating voluntary ethanol consumption.

It is an object of the present invention to identify methods of inhibiting alcohol consumption comprising administering a selective melanocortin 4 receptor agonist to a subject. It is another object of the present invention to identify methods of treating alcoholism and alcohol abuse comprising administering a selective melanocortin 4 receptor agonist to a subject. It is yet another object of the invention to identify methods of preventing alcoholism, alcohol abuse, and alcohol-related disorders. It is a further object of the present invention to provide a method of manufacture of a medicament useful to inhibit alcohol consumption.

### SUMMARY OF THE INVENTION

The present invention provides a method of inhibiting alcohol consumption comprising administration of a selective melanocortin 4 receptor agonist to a subject.

The present invention further provides a method of reducing alcohol consumption comprising administration of a selective melanocortin 4 receptor agonist to a subject.

The present invention further provides a method of preventing alcohol consumption comprising administration of a selective melanocortin 4 receptor agonist to a subject.

The present invention also provides a method of treating or preventing alcoholism comprising administration of a selective melanocortin 4 receptor agonist to a subject.

The present invention also provides a method of treating or preventing alcohol abuse comprising administration of a selective melanocortin 4 receptor agonist to a subject.

The present invention further provides a method of treating or preventing alcohol-related diseases comprising administration of a selective melanocortin 4 receptor agonist to a subject.

The present invention is also concerned with treatment of these conditions, and the use of the compositions of the present invention for manufacture of a medicament useful for treating these conditions.

### BRIEF DESCRIPTION OF THE DRAWINGS

FIG. 1(a). Shows the 8 day average consumption (g/kg/day) of ethanol solutions (containing 3%, 1%, 10%, and 20% of ethanol) by mice lacking the melanocortin 3 receptor (Mc3r^{-/-}), compared to consumption by littermate wild-type (Mc3r^{+/+}) mice maintained on an inbred C57BL/6J genetic background.

FIG. 1(b). Shows the ethanol preference ratios (volume of ethanol consumed/total volume of fluid consumed) as a measure of relative ethanol preference of mice lacking the melanocortin 3 receptor (Mc3r^{-/-}), compared to the ethanol preference ratio of littermate wild-type (Mc3r^{+/+}) mice maintained on an inbred C57BL/6J genetic background based on the ethanol consumption volumes of FIG. 1(a).

FIG. 2(a). Shows the effect of consumption of 20% ethanol (g/kg/2-h) over 2 hours by C57BL/6J mice following intracerebroventricular (i.c.v.) infusion of MC4R agonist cyclo(NH—CH₂—CH₂—CO-His-D-Phe-Arg-Trp-Glu)-NH₂ (compound A)(1.0 or 3.0 μg), compared to C57BL/6J mice following an intracerebroventricular (i.c.v.) infusion of aCSF (cerebral spinal fluid).

### DETAILED DESCRIPTION OF THE INVENTION

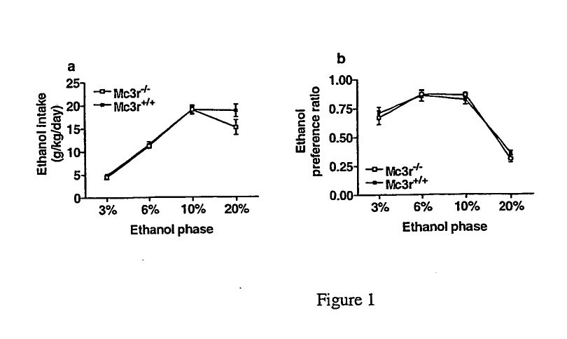

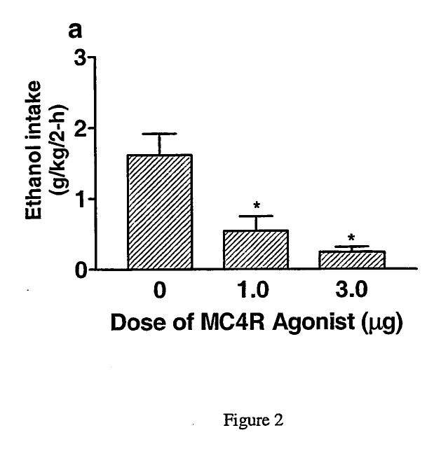

In rodents, melanocortin peptides act through at least five receptor subtypes, namely MC1R, MC2R, MC3R, MC4R, and MC5R, all of which couple to heterotrimeric G-proteins that stimulate adenylyl cyclase activity. Melanocortin receptors in the rodent brain are primarily comprised of the MC3R and MC4R subtypes, but MC1R and MC5R are detected at low levels and in limited regions. The present invention shows that selective melanocortin 4 receptor agonists inhibit alcohol consumption. It was found that C57BL/6J mice treated with a selective melanocortin-4 receptor agonist, cyclo(NH—CH₂—CH₂—CO-His-D-Phe-Arg-Trp-Glu)-NH₂ (Compound A), consume up to 86% less alcohol than untreated controls (See FIG. 2(a)). While there were no significant differences between the mice lacking the melanocortin 3 receptor (Mc3r^{-/-}) and the littermate wild-type (Mc3r^{+/+}) mice in voluntary ethanol consumption (See FIGS. 1(a) and (b)), subsequent treatment of C57BL/6J mice with compound A resulted in a significant reduction of ethanol drinking in the C57BL/6J mice.

Compound A is a highly selective agonist for MC4R with 90-fold selectivity over MC3R and greater than 2000-fold selectivity over MC5R, and shows extremely weak binding and activation of MC5R (Bednarek et al., 2001).

The present invention provides a method of inhibiting alcohol consumption. The present invention further provides a method of reducing alcohol consumption. The present invention further provides a method of treating or preventing alcoholism. The present invention further provides a method of treating or preventing alcohol abuse. The present invention also provides a method of treating or preventing alcohol-related disorders. The present invention also relates to pharmaceutical compositions, and medicaments useful for carrying out these methods.

The compositions of the present invention comprise a melanocortin 4 receptor agonist. The melanocortin 4 receptor agonist of use in the present invention may be any melanocortin 4 receptor agonist known in the art. For convenience, the use of an orally active melanocortin 4 receptor agonist is preferred.

In one embodiment of the present invention, the melanocortin 4 receptor agonists useful in the present invention are represented by the compounds of structural Formula I: 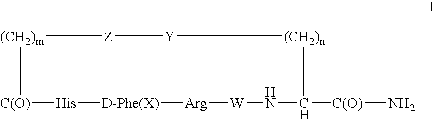
wherein,
His is L-histidyl;
D-Phe(X) is D-phenylalanyl unsubstituted or optionally para-substituted with a group selected from F, Cl, Br, Me, OMe;
Arg is L-arginyl;
W is L-tryptophanyl or 2-naphthyl-L-alanyl;
one of Y and Z is —C(O)—and the other is —NH—;
mis 1 to 4;
n is 1 to 4, provided that n+m is 4 to 6; or a pharmaceutically acceptable salt thereof.

In one embodiment of Formula I, Z is —C(O)—and Y is —NH—. In one subset thereof, m is 2. In another subset thereof, n is 2 to 4. In another subset thereof, D-Phe(X) is D-phenylalanyl optionally para-substituted with chlorine.

In another embodiment Y is —C(O)— and Z is —NH—. In one subset thereof n is 2. In another subset thereof m is 2 to 4. Another subset thereof provides compounds where W is L-tryptophanyl and D-Phe(X) is D-phenylalanyl.

In a sub-class of this class, the MC4R agonist is selected from the group consisting of: cyclo(NH—CH₂—CH₂—CO-His-D-Phe-Arg-Trp-Glu)-NH₂, and pharmaceutically acceptable salts thereof.

Specific examples of compounds of Formula I are shown in the following Table:

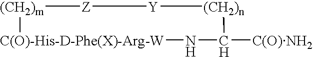

| Example | Z | Y | X | W | m | n |
|---|---|---|---|---|---|---|
| 1 | C(O) | NH | H | Trp | 2 | 4 |
| 2 | C(O) | NH | H | Trp | 2 | 2 |
| 3 | C(O) | NH | H | Trp | 2 | 1 |
| 4 | C(O) | NH | Para-Cl | Trp | 2 | 4 |
| 5 | C(O) | NH | H | 2-Nal | 2 | 4 |
| 6 | NH | C(O) | H | Trp | 4 | 2 |
| 7 | NH | C(O) | H | Trp | 3 | 2 |
| 8 | NH | C(O) | H | Trp | 2 | 2 |
| 9 | NH | C(O) | H | Trp | 1 | 2 |

The melanocortin 4 receptor agonists of Formula I, including cyclo(NH—CH₂—CH₂—CO-His-D-Phe -Arg-Trp-Glu)-NH₂ (Compound A), the compounds of Examples 1-9, and their preparation are disclosed in WO 03/006604, which is hereby incorporated by reference in its entirety.

One of ordinary skill in the art, can readily identify MC4R agonist compounds useful in the compositions and methods of the present invention using the methods described in Bednarek et al., Peptides, 20 (1999) 401-409). MC4R agonists which are useful in the present invention generally have an IC₅₀ less than 100 nM in the MC4R agonist binding assay described in Bednarek et al., Peptides, 20 (1999) 401-409).

In one embodiment of the present invention, the invention is directed to a method of inhibiting alcohol consumption comprising administering to a subject a therapeutically effective amount of a melanocortin 4 receptor agonist, or a pharmaceutically acceptable salt thereof, wherein the functional activity of the melanocortin 4 receptor agonist is characterized by an EC₅₀ at least 15-fold more selective for the human melanocortin 4 receptor than for the human melanocortin 1 receptor, the human melanocortin 3 receptor and the human melanocortin 5 receptor.

In a class of this embodiment, the functional activity of the melanocortin 4 agonist is characterized by an EC₅₀ at least 17-fold more selective for the human melanocortin 4 receptor than for the human melanocortin 3 receptor.

In another class of this embodiment, the functional activity of the melanocortin 4 agonist is characterized by an EC₅₀ at least 90-fold more selective for the human melanocortin 4 receptor than for the human melanocortin 3 receptor.

In another class of this embodiment, the functional activity of the melanocortin 4 agonist is characterized by an EC₅₀ at least 200-fold more selective for the human melanocortin 4 receptor than for the human melanocortin 5 receptor.

In another class of this embodiment, the functional activity of the melanocortin 4 agonist is characterized by an EC₅₀ at least 3000-fold more selective for the human melanocortin 4 receptor than for the human melanocortin 5 receptor.

In another embodiment of the present invention, the invention is directed to a method of inhibiting alcohol consumption comprising administering to a subject a therapeutically effective amount of a melanocortin 4 receptor agonist, or a pharmaceutically acceptable salt thereof, wherein the binding affinity of the melanocortin 4 receptor agonist is characterized by an IC₅₀ at least 25-fold more selective for the human melanocortin 4 receptor than for the human melanocortin 3 receptor and the human melanocortin 5 receptor.

In a class of this embodiment, the binding affinity of the melanocortin 4 agonist is characterized by an IC₅₀ at least 50-fold more selective for the human melanocortin 4 receptor than for the human melanocortin 3 receptor.

In another class of this embodiment, the binding affinity of the melanocortin 4 agonist is characterized by an IC₅₀ at least 100-fold more selective for the human melanocortin 4 receptor than for the human melanocortin 3 receptor.

In another class of this embodiment, the binding affinity of the melanocortin 4 agonist is characterized by an IC₅₀ at least 50-fold more selective for the human melanocortin 4 receptor than for the human melanocortin 5 receptor.

In another class of this embodiment, the binding affinity of the melanocortin 4 agonist is characterized by an IC₅₀ at least 1000-fold more selective for the human melanocortin 4 receptor than for the human melanocortin 5 receptor.

In another embodiment of the present invention, the invention is directed to a method of preventing alcohol consumption, alcoholism, alcohol abuse or an alcohol related disorder comprising administering a selective melanocortin 4 receptor agonist, or a pharmaceutically acceptable salt thereof, to a subject wherein the selective melanocortin 4 receptor agonist is a compound of Formula I: 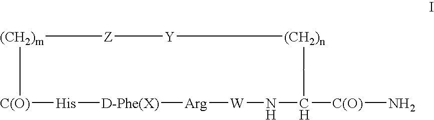
wherein:
His is L-histidyl;
D-Phe(X) is D-phenylalanyl optionally para-substituted with a group selected from F, Cl, Br, Me, and OMe;
Arg is L-arginyl;
W is L-tryptophanyl or 2-naphthyl-L-alanyl;
one of Y and Z is —C(O)— and the other is —NH—;
m is 1 to 4; and
   n is 1 to 4, provided that n+m is 4 to 6. In a class of this embodiment, the alcohol related disorder is selected from the group consisting of: liver disease; hepatitis; inflammation of the liver; alcoholic cirrhosis; heart disease; high blood pressure; stroke; esophageal cancer; mouth cancer; throat cancer; voice box cancer; breast cancer; colon cancer; rectal cancer; pancreatitis; alcoholic dementia; Wernicke-Korsakoff syndrome; brain damage; slow bone healing; impaired wound healing; and diminished immune defenses.

In another embodiment of the present invention, the invention is directed to the use of a therapeutically effective amount of a melanocortin 4 receptor agonist of Formula I: 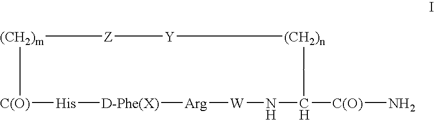
wherein:
His is L-histidyl;
D-Phe(X) is D-phenylalanyl optionally para-substituted with a group selected from F, Cl, Br, Me, and OMe;
Arg is L-arginyl;
W is L-tryptophanyl or 2-naphthyl-L-alanyl;
one of Y and Z is —C(O)— and the other is —NH—;
mis 1 to 4;
n is 1 to 4, provided that n+m is 4 to 6; or
a pharmaceutically acceptable salt thereof;
   for the manufacture of a medicament useful to prevent an alcohol related disorder in a subject. In a class of this embodiment, the alcohol related disorder is selected from the group consisting of: liver disease; hepatitis; inflammation of the liver; alcoholic cirrhosis; heart disease; high blood pressure; stroke; esophageal cancer; mouth cancer; throat cancer; voice box cancer; breast cancer; colon cancer; rectal cancer; pancreatitis; alcoholic dementia; Wernicke-Korsakoff syndrome; brain damage; slow bone healing; impaired wound healing; and diminished immune defenses.

The above compounds are only illustrative of the MC4R agonists that can be used in the compositions of the present invention. As this listing of compounds is not meant to be comprehensive, the methods of the present invention may employ any MC4R agonists, including the MC4R agonists of Formulas I-VI, and are not limited to any particular structural class of compounds.

The term “2-Nal” refers to 2-naphthyl-L-alanyl.

The term “pharmaceutically acceptable salts” refers to the pharmaceutically acceptable and common salts, for example, a base addition salt to carboxyl group when the compound has a carboxyl group, or an acid addition salt to amino or basic heterocyclyl when the compound has an amino or basic heterocyclyl group, including quaternary ammonium salts, prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylarninoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, trifluoro acetate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to MC4R agonists, and MC4R agonists of Formula I, are meant to also include the pharmaceutically acceptable salts and esters thereof.

The pharmaceutically acceptable salts of the composition of the instant invention include the composition wherein one of the individual components of the composition is in the form of a pharmaceutically acceptable salt, or the composition wherein all of the individual components are in the form of pharmaceutically acceptable salts (wherein the salts for each of the components can be the same or different), or a pharmaceutically acceptable salt of the combined components (i.e., a salt of the composition).

The “pharmaceutically acceptable esters” in the present invention refer to non-toxic esters, for example, the pharmaceutically acceptable, common esters on carboxyl group when the compound has a carboxyl group, for example, esters with lower alkyls (for example methyl, ethyl, propyl, isopropyl, butyl, sec-butyl, tert-butyl, pentyl, isopentyl, neopentyl, cyclopropyl, cyclobutyl, cyclopentyl), aralkyls (for example benzyl, phenethyl), lower alkenyls (for example allyl, 2-butenyl), lower alkoxy (lower) alkyls (for example methoxymethyl, 2-methoxyethyl, 2-ethoxyethyl), lower alkanoyloxy (lower) alkyls (for example acetoxymethyl, pivaloyloxy-methyl, 1-pivaloyloxyethyl), lower alkoxycarbonyl (lower) alkyls (for example methoxycarbonylmethyl, isopropoxycarbonylmethyl), carboxy-(lower)alkyls (for example carboxymethyl), lower alkoxycarbonyloxy-(lower)alkyls (for example 1-(ethoxycarbonyloxy)ethyl, 1-(cyclohexyl-oxycarbonyloxy)ethyl), carbamoyloxy(lower)alkyls (for example carbamoyloxymethyl), phthalidyl group, (5-substituted-2-oxo-1,3-dioxol-4-yl)methyl (for example (5-methyl-2-oxo-1,3-dioxol-4-yl)methyl), and the like.

The compounds in the compositions of the present invention include stereoisomers, such as optical isomers, diastereomers and geometerical isomers, or tautomers depending on the mode of substitution. The compounds may contain one or more chiral centers and occur as racemates, racemic mixtures and as individual diastereomers, enantiomeric mixtures or single enantiomers, or tautomers, with all isomeric forms being included in the present invention. The present invention is meant to comprehend all such isomeric forms of the compounds in the compositions of the present invention, and their mixtures. Therefore, where a compound is chiral, the separate enantiomers, substantially free of the other, are included within the scope of the invention; further included are all mixtures of the two enantiomers. Also included within the scope of the invention are polymorphs, hydrates and solvates of the compounds of the instant invention.

The present invention includes within its scope prodrugs of the compounds in the compositions of this invention. In general, such prodrugs will be functional derivatives of the compounds in these compositions which are readily convertible in vivo into the required compound. Thus, in the methods of treatment of the present invention, the term “administering” shall encompass the treatment of alcoholism, alcohol abuse, alcohol consumption and alcohol related disorders with the compounds specifically disclosed as elements of the composition or with compounds which may not be specifically disclosed, but which convert to the specified compounds in vivo after administration to the patient. Conventional procedures for the selection and preparation of suitable prodrug derivatives are described, for example, in “Design of Prodrugs,” ed. H. Bundgaard, Elsevier, 1985.

The compounds of the present invention are useful to inhibit or reduce voluntary alcohol consumption, and for the treatment or prevention of alcoholism, alcohol abuse, and alcohol-related disorders.

Alcoholism, also known as alcohol dependence, is a disease that is characterized by abnormal alcohol seeking behavior that leads to impaired control over drinking. Alcoholism may include some or all of the following symptoms: narrowing of drinking repertoire (drinking only one brand or type of alcoholic beverage); craving (a strong need or urge to drink), loss of control (not being able to stop drinking once drinking has begun), drink seeking behavior (attending only social events that include drinking); physical dependence (withdrawal symptoms, such as nausea, sweating, shakiness, and anxiety after cessation of drinking), drinking to relieve or avoid withdrawal symptoms; and tolerance (the need to drink greater amounts of alcohol to achieve previous effects); subjective awareness of the compulsion to drink or craving for alcohol; and relapse (a return to drinking after a period of abstinence).

Alcohol abuse is a pattern of drinking that results in one or more of the following situations within a 12 month period: failure to fulfill major work, school or home responsibilities; drinking in situations that are physically dangerous, such as while driving a car or operating machinery; having recurring alcohol related legal problems, such as being arrested for driving under the influence of alcohol, or physically hurting someone while drunk; and continued drinking despite ongoing relationship problems that are caused or worsened by the drinking. Harmful alcohol use implies alcohol use that causes health consequences, such as physical or mental damage. Alcohol related disorders include, but are not limited to, disorders resulting from alcohol dependence, alcohol abuse and alcohol consumption. Alcohol related disorders include, but are not limited to: liver disease, such as hepatitis, inflammation of the liver, and alcoholic cirrhosis; heart disease; high blood pressure; stroke; certain forms of cancer, such as esophageal, mouth, throat, voice box, breast, colon and rectal cancer; pancreatitis; alcoholic dementia, Wernicke-Korsakoff syndrome, brain damage, and death. Alcohol misuse has also been found to predispose the subject to osteoporosis, slow bone healing, impaired wound healing, inhibited osteoblastic function and diminished immune defenses. Alcohol intoxication increases the risk of further accidents, and decreases the pain inhibition that would make a normal patient more careful. Alcohol dependence also leads to altered cognitive and emotional functions, and thought processes, such as impaired judgment, feelings of incompetency, low self-esteem, despair in relationships, depression, and feelings of failure. “Treatment” (of alcoholism or alcohol abuse) refers to the administration of the compounds or combinations of the present invention to reduce or inhibit the consumption of alcohol in a subject. One outcome of treatment may be reducing the consumption of alcohol in a subject relative to the subject's alcohol consumption prior to treatment. Another outcome of treatment may be inhibiting consumption of alcohol in a subject. Another outcome of treatment may be decreasing the occurrence of alcohol intake in a subject. Another outcome of treatment may be decreasing the severity of alcohol intake, such as decreasing the amount of alcohol consumed, in a subject. Another outcome of treatment may be to administer the compounds or combinations of the present invention to reduce or inhibit the consumption of alcohol in a subject in need thereof.

The term “inhibit” alcohol consumption means to stop alcohol consumption in a subject. One outcome of inhibition may be to stop alcohol consumption in a subject in need thereof.

The term “reduce” alcohol consumption means to decrease the amount of alcohol consumed by a subject relative to the amount of alcohol consumed prior to the start of treatment. In one embodiment the amount of alcohol consumed by a subject is decreased by at least 10% relative to the amount of alcohol consumed prior to the start of treatment. In another embodiment, the amount of alcohol consumed by a subject is decreased by at least 25% relative to the amount of alcohol consumed prior to the start of treatment. In another embodiment, the amount of alcohol consumed by a subject is decreased by at least 67% relative to the amount of alcohol consumed prior to the start of treatment. In yet another embodiment, the amount of alcohol consumed by a subject is decreased by at least 86% relative to the amount of alcohol consumed prior to the start of treatment.

“Prevention” (of alcoholism) refers to the administration of the compounds or combinations of the present invention to prevent alcohol intake, alcohol consumption, alcohol abuse, alcoholism or developing an alcohol-related disorder in a subject. One outcome of prevention may be to prevent alcohol intake in a subject. Another outcome of prevention may be to prevent alcohol abuse in a subject. Another outcome of prevention may be to prevent alcoholism in a subject. Another outcome of prevention may be to prevent the development of an alcohol-related disorder in a subject. Another outcome of prevention may be preventing alcohol consumption from occurring if the treatment is administered prior to the onset of alcohol consumption in a subject. Another outcome of prevention may be to prolong resistance to alcohol consumption in a subject. Another outcome of prevention may be to administer the compounds or combinations of the present invention to prevent alcohol intake in a subject at risk of alcohol consumption, alcohol abuse, alcoholism or developing an alcohol-related disorder in a subject.

Moreover, if treatment is commenced in a subject already consuming alcohol, such treatment may prevent the occurrence, progression or severity of alcohol-related disorders, such as, but not limited to, liver disease; hepatitis; inflammation of the liver; alcoholic cirrhosis; heart disease; high blood pressure; stroke; esophageal cancer, mouth cancer; throat cancer; voice box cancer; breast cancer; colon cancer; rectal cancer; pancreatitis; alcoholic dementia; Wernicke-Korsakoff syndrome; brain damage; osteoporosis; slow bone healing; impaired wound healing; diminished immune defenses; depression; and death.

The term “alcohol” is understood to mean ethanol.

The terms “selective” melanocortin 4 receptor agonist and “selective melanocortin 4 receptor agonist” should be understood to mean a melanocortin 4 receptor agonist with a functional activity at the human melanocortin 4 receptor that is characterized by an EC₅₀ at least 15-fold lower than the EC₅₀ of the melanocortin 4 receptor agonist at the human melanocortin 1 receptor, the human melanocortin 3 receptor and the human melanocortin 5 receptor.

In one embodiment of the present invention, the functional activity of the melanocortin 4 agonist at the human melanocortin 4 receptor is characterized by an EC₅₀ at least 17-fold lower than the EC₅₀ of the melanocortin 4 receptor agonist at the human melanocortin 3 receptor.

In another embodiment of the present invention, the functional activity of the melanocortin 4 agonist at the human melanocortin 4 receptor is characterized by an EC₅₀ at least 90-fold lower than the EC₅₀ of the melanocortin 4 receptor agonist at the human melanocortin 3 receptor.

In another embodiment of the present invention, the functional activity of the melanocortin 4 agonist at the human melanocortin 4 receptor is characterized by an EC₅₀ at least 200-fold lower than the EC₅₀ of the melanocortin 4 receptor agonist at the human melanocortin 5 receptor.

In another embodiment of the present invention, the functional activity of the melanocortin 4 agonist at the human melanocortin 4 receptor is characterized by an EC₅₀ at least 3000-fold lower than the EC₅₀ of the melanocortin 4 receptor agonist at the human melanocortin 5 receptor.

The terms “administration of” and or “administering” a compound should be understood to mean providing a compound of the invention or a prodrug of a compound of the invention to a subject. The instant pharmaceutical composition includes administration of a single pharmaceutical dosage formulation which contains the melanocortin 4 receptor agonist.

The term “subject”, as used herein refers to an animal, preferably a mammal, more preferably a human. In one embodiment of the present invention, the term “subject” refers to a human that is or has been the object of treatment, observation or experiment. In another embodiment of the present invention, the term “subject” refers to a “subject in need thereof”. In a class of this embodiment, the “subject in need thereof” refers to a subject who is in need of treatment or prophylaxis as determined by a researcher, veterinarian, medical doctor or other clinician. In another embodiment, the “subject in need thereof” is a human that is alcohol dependent. In another embodiment, the “subject in need thereof” is a human that is an alcoholic. In another embodiment, the “subject in need thereof” is a human that abuses alcohol. In another embodiment, the “subject in need thereof” is a human that consumes alcohol. In another embodiment, the “subject in need thereof” has an alcohol-related disorder. In yet another embodiment of the present invention, the term “subject” refers to a “subject at risk thereof”. In a class of this embodiment, the “subject at risk thereof” is a subject at risk of developing alcoholism. In another class of this embodiment, the “subject at risk thereof” is a subject at risk of developing an alcohol-related disorder.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated. Disorders being treated include, but are not limited to, alcohol dependence, alcoholism, or alcohol abuse, or alcohol consumption or an alcohol-related disorder in subjects in need thereof. The novel methods of treatment of this invention are for disorders known to those skilled in the art.

The term “prophylactically effective amount” as used herein means the amount of the active compounds that will elicit the biological or medical response in a tissue, system, subject, or human that is being sought by the researcher, veterinarian, medical doctor or other clinician, to prevent the onset of alcohol dependence, alcoholism, or alcohol abuse, or alcohol consumption or an alcohol-related disorder in subjects as risk for alcohol dependence, alcoholism, alcohol abuse, alcohol consumption or an alcohol-related disorder.

The magnitude of prophylactic or therapeutic dose of the active ingredient (the MC4R agonist) will, of course, vary with the nature of the severity of the condition to be treated and with the particular compound and its route of administration. It will also vary according to the age, weight and response of the individual patient. In general, the daily dose range of each compound lies within the range of from about 0.0001 mg/kg to about 1000 mg/kg; more specifically from about 0.001 mg/kg to about 1000 mg/kg body weight of a subject per day in single or divided doses. On the other hand, it may be necessary to use dosages outside these limits in some cases.

For use where a composition for intravenous administration is employed, a suitable dosage range is from about 0.0001 mg/kg to about 1000 mg/kg; more specifically from 0.001 mg/kg to about 100 mg/kg of each compound in the composition per day.

In the case where an oral composition is employed, a suitable dosage range is, e.g. from about 0.0001 mg/kg to about 1000 mg/kg of each compound in the composition per day; more specifically from about 0.001 mg to about 1000 mg per day. For oral administration, the compositions are provided in the form of tablets containing from 0.01 mg to 1,000 mg; more specifically 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient for the symptomatic adjustment of the dosage to the subject to be treated. This dosage regimen may be adjusted to provide the optimal therapeutic response.

The compounds of this invention can be administered to humans in the dosage ranges specific for each compound. In general, for treating alcoholism, alcohol abuse, alcohol consumption and/or an alcohol related disorder, the MC4R agonist is administered at a daily dosage of from about 0.0001 mg/kg to about 1000 mg/kg of body weight orally. More specifically, when treating alcoholism, alcohol abuse, alcohol consumption and/or an alcohol related disorder, generally satisfactory results may be obtained when a MC4R agonist, such as a MC4R agonist of Formula I, or a pharmaceutically acceptable salt or ester thereof, is administered at a daily oral dosage of from about 0.001 mg/kg to about 1000 mg/kg; more specifically from about 0.001 mg/kg to about 100 mg/kg of body weight, given in a single dose or in divided doses two to six times a day, or in sustained release form.

The effective dosage of the active ingredient employed in the composition may vary depending on the particular compound employed, the mode of administration, the condition being treated and the severity of the condition being treated. Thus, the dosage regimen utilizing the compositions of the present invention is selected in accordance with a variety of factors including type, species, age, general health, body weight, diet, sex and medical condition of the subject; the severity of the condition to be treated; the renal and hepatic function of the patient; the drug combination; and the particular compound employed and its route of administration. A physician, clinician or veterinarian of ordinary skill can readily determine and prescribe the effective amount of the drug required to prevent, counter or arrest the progress of the condition.

Another aspect of the present invention provides pharmaceutical compositions comprising a pharmaceutical carrier and a therapeutically effective amount of each compound in the composition of the present invention. The term “composition”, as in pharmaceutical composition, is intended to encompass a product comprising the active ingredient(s), and the inert ingredient(s), such as pharmaceutically acceptable excipients, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a MC4R agonist, additional active ingredient(s), and/or pharmaceutically acceptable excipients and carriers.

Any suitable route of administration may be employed for providing a subject, especially a human, with an effective dosage of a composition of the present invention. For example, oral delivery, rectal delivery, topical delivery, parenteral delivery, ocular delivery, pulmonary delivery, nasal delivery, delivery to the central nervous system, in particular the brain, and the like may be employed. Dosage forms include tablets, troches, dispersions, suspensions, solutions, capsules, creams, ointments, aerosols, and the like.

The pharmaceutical compositions of the present invention comprise a MC4R agonist, as active ingredient or a pharmaceutically acceptable salt or ester thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compounds suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. These compositions may be conveniently presented in unit dosage form and prepared by any of the methods well-known in the art of pharmacy.

For administration by inhalation, the compositions of the present invention are conveniently delivered in the form of an aerosol spray presentation from pressurized packs or nebulizers. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients. Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will, of course be continuous rather than intermittent throughout the dosage regimen. The compositions of the present invention can also be administered in the form of liposome delivery systems, such as small unilamellar vesicles, large unilamellar vesicles and multilamellar vesicles. Liposomes can be formed from a variety of phospholipids, such as cholesterol, sterylamine or phosphatidylcholines. Compositions of the present invention may also be delivered by the use of monoclonal antibodies as individual carriers to which the compound molecules are coupled. The compounds in these compositions may also be coupled with soluble polymers as targetable drug carriers. Such polymers can include polyvinylpyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

In practical use, each compound in the compositions of the present invention (e.g. MC4R agonist) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200 and 4,008,719, which are hereby incorporated by reference in their entirety.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta4actose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil. The active compounds can also be administered intranasally as, for example, liquid drops or spray.

Desirably, each tablet contains from 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active ingredient in the composition of the present invention (e.g. MC4R agonist) for the symptomatic adjustment of the dosage to the subject to be treated; and each cachet or capsule contains from about 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 750, 850 and 1,000 milligrams of each active in the composition of the present invention (e.g. MC4R agonist) for the symptomatic adjustment of the dosage to the subject to be treated.

Exemplifying the invention is a pharmaceutical composition comprising a MC4R agonist described above and a pharmaceutically acceptable carrier. Also exemplifying the invention is a pharmaceutical composition made by combining any of the MC4R agonists described above and a pharmaceutically acceptable carrier. An illustration of the invention is a process for making a pharmaceutical composition comprising combining any of the MC4R agonists described above and a pharmaceutically acceptable carrier.

The dose may be administered in a single daily dose or the total daily dosage may be administered in divided doses of two to six times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compositions of the present invention:

| | mg/ | |
|---|---|---|
| Tablet | tablet | |
| cyclo(NH—CH₂—CH₂—CO-His-D-Phe-Arg-Trp-Glu)-NH₂ | 25 | |
| (compound A) | | |
| Microcrystalline Cellulose | 50.5 | |
| Lactose | 111.5 | |
| Croscarmellose Sodium | 5.0 | |
| Hydroxypropylcellulose | 6.0 | |
| Sodium Dodecyl Sulfate | 1.0 | |
| Magnesium Stearate | 1.0 | |
| | 200 | |
| | mg/ | |
| Capsule | capsule | |
| cyclo(NH—CH₂—CH₂—CO-His-D-Phe-Arg-Trp-Glu)-NH₂ | 100 | |
| (compound A) | | |
| Lactose | 80 | |
| Sodium Dodecyl Sulfate | 20 | |
| | 200 | |
| | Per | |
| Aerosol | canister | |
| cyclo(NH—CH₂—CH₂—CO-His-D-Phe-Arg-Trp-Glu)-NH₂ | 13 | mg |
| (compound A) | | |
| Lecithin, NF Liq. Conc. | 1.2 | mg |
| Trichlorofluoromethane, NF | 4.025 | g |
| Dichlorodifluoromethane, NF | 12.15 | g |

It will be understood that the scope of compositions of the compounds of this invention with other agents useful to inhibit or reduce alcohol consumption and for treating or preventing alcoholism, alcohol abuse, and alcohol related conditions includes in principle any combination with any pharmaceutical composition useful to inhibit or reduce alcohol consumption and for treating alcoholism, alcohol abuse and alcohol related disorders.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### EXAMPLE 1

Competitive Binding Assay

Materials and Methods

The peptides of the present invention were evaluated for agonist activity in receptor binding assay. Crude membrane preparations were obtained from Chinese hamster ovary cells expressing human MC3, MC4, and MC5 receptors. Cells were rinsed with phosphate-buffered saline (PBS) lacking CaCl₂ or MgCl₂ (Life Technologies, Gaithersburg, Md., USA), and then detached with enzyme-free dissociation media (Specialty Media, Lavellette, N.J., USA). Cells were pelleted at 2800×g for 10 minutes and resuspended in membrane buffer (20 mM Tris, pH 7.2, 5 mM ethylenediaminetetraacetic acid) with 5 μg/ml leupeptin, 5 μg/ml aprotinin, 40 μg/ml bacitracin, and 25 μg/m pefabloc (Boehringer Mannheim). The cells were doused with 10 strokes by using a motor-driven Teflon-coated pestle in a glass homogenizer at low speed. The resulting cell suspension was centrifuged at 4100×g, 4° C., for 20 minutes. The pellet was resuspended in fresh membrane buffer with protease inhibitors, aliquoted, snap-frozen in liquid nitrogen, and stored at −80° C. The resulting crude membranes were titrated to determine the optimal level necessary for performing binding studies. Results

Binding reactions (total volume=250 μg) contained MBB (50 mM Tris, pH 7.2, 2 mM CaCl_{2, 1} mM MgCl₂), 0.1% bovine serum albumin, crude membranes prepared from cells expressing human MC3, MC4, or MC5 receptor, 200 pM of [125I]-NDP-α-MSH (Amersham, Arlington Heighs, Ill., USA), and increasing concentrations of unlabeled test compounds dissolved in dimethylsulfoxide (final concentration=2%). Reactions were incubated for 1 hour without shaking and then filtered through 96-well filter plates (Packard), presoaked in 1% polyethyleneimine. Filters were washed 3 times with TNE buffer (50 mM Tris, pH 7.4, 5 mM ethylenediaminetetraacetic acid, 150 mM NaCl), dried and counted by using Microscint-20 in a Topcount scintillation counter (Packard). Nonspecific binding was determined in the presence of 2 μm of unlabeled NDP-α-MSH (Peninsula Laboratories). Binding data were analyzed with GraphPad curve-fitting software (PRISM, San Diego, Calif.) and are presented in the Table below. Active peptides were evaluated in three independent experiments.

Results of binding assay (Example 1) and selectivity for representative compounds of the present invention are provided below:

| | Binding Assay IC₅₀ (nM) | | | Selectivity Binding IC₅₀ | |
|---|---|---|---|---|---|
| Ex | hMC-3R | hMC-4R | hMC-5R | 3R/4R | 5R/4R |
| 1 | 418 | 25 | 3103 | 16.7 | 124 |
| 2 | 1800 | 35 | 7200 | 51.3 | 205 |
| 3 | 1600 | 71 | 3600 | 22.5 | 50.7 |
| 4 | 17 | 1.7 | 92 | 10 | 54 |
| 5 | 440 | 13 | >20000 | 33.85 | 1538 |
| 6 | 580 | 12 | 9000 | 48 | 750 |
| 7 | 1830 | 41 | >5000 | 44 | 121.9 |
| 8 | 490 | 4 | 4600 | 122.5 | 1150 |
| 9 | >1000 | 290 | >1000 | 3.45 | 3.45 |

### EXAMPLE 2

cAMP Assays

Materials and Methods

Chinese hamster ovary cells expressing a human melanocortin receptor were rinsed with calcium-and magnesium-free PBS (Life Technologies), and detached from the tissue culture flasks by 5-minutes incubation with enzyme-free dissociation buffer (S-014-B, Specialty Media). Cells were collected by centrifugation and resuspended in Earle's balanced salt solution (Life Technologies) with addition of 10 mM 4-(2-hydroxyethyl)-1-piperazineethanesulfonic acid (Hepes) buffer, pH 7.5, 5 mM MgCl2, 1 mM glutamine, and 1 mg/ml bovine serum albumin to concentration of 1-5×10⁶ cells/ml. Subsequently, cells were counted and the cell suspension was treated with the phosphodiesterase inhibitor 3-isobutyl-1-methylxanthine (to concentration of 0.6 mM).

A test compound was dissolved in dimethyl sulfoxide (DMSO, 10⁻³ to 10⁻⁸ M), diluted with buffer and 0.1 volume of the solution was added to 0.9 volumes of the cell suspension (1 to 5×10⁵ cells); final concentration of DMSO was 1%. After 45 minutes at room temperature, cells were lysed by incubation at 100° C. for 5 minutes to release accumulated cAMP. Accumulation of cAMP was measured in an aliquot of the cell lysate with the Amersham (Arlington Heights, Ill.) cAMP detection assay kit (RPA556). The amount of cAMP produced in response to a tested compound was compared to the amount of cAMP produced in response to α-MSH, defined as a 100% agonist. All active peptides were characterized in three independent experiments.

Results

Results of cAMP functional binding assay (Example 2) and selectivity for representative compounds of the present invention are provided below:

| | cAMP Assay EC₅₀ (nM) | | | | Selectivity cAMP EC₅₀ | | |
|---|---|---|---|---|---|---|---|
| Ex | hMC-1R | hMC-3R | hMC-4R | hMC-5R | 1R/4R | 3R/4R | 5R/4R |
| 1 | | 110 | 3.3 | 1180 | | 33 | 357 |
| 2 | | 240 | 2.9 | 2200 | | 83 | 758 |
| 3 | | 590 | 33 | 12% @ 5 | | 17.8 | |
| 4 | | 40 | 0.74 | 170 | | 54 | 229.7 |
| 5 | | 360 | 3.7 | >5000 | | 97.3 | 1351.3 |
| 6 | | 190 | 2.7 | 1900 | | 70.4 | 703.7 |
| 7 | | 310 | 5.7 | >5000 | | 54.39 | 877 |
| 8 | 11 | 59 | 0.53 | 1900 | 20.75 | 111 | 3585 |
| 9 | | 2200 | 35 | 15% @ 5 | | 62.86 | |

### EXAMPLE 3

Voluntary Ethanol Consumption

Materials and Methods

The generation of Mc3r^{-/-} mice has been described (Chen, S. et al., Nature Genetics (2000) 26: 97-102). Mc3r^{-/-} mice are born at the expected frequency and are viable and fertile. Gross anatomical and histological assessment of these mice revealed no abnormalities of brain or other organs; the Mc3r^{-/-} mice have increased fat mass, decreased lean mass, and are hypophagic. Studies described below utilized male and female Mc3r^{-/-} and/or Mc3r^{+/+} mice of a C57BL/6J genetic background, 8 to 12 weeks of age at the start of experiment. Non-littermate heterozygous (Mc3r^{+/-}) mice were bred, resulting in Mc3r^{-/-} and Mc3r^{+/+}F2 littermate mice. Mice were individually housed in polypropylene cages with wood-chip bedding and had ad libitum access to standard rodent chow (Teklad, Madison, Wis.) and water throughout the experiments except where noted. The colony room was maintained at approximately 22° C. with a 12h: 12h light:dark cycle. All procedures used in the present research were in compliance with the National Institute of Health guidelines, and the protocols were approved by the University of North Carolina Animal Care and Use Committee.

Throughout the experiments, fluid intake was assessed every 2 days and body weights were recorded weekly. Mc3r^{-/-} (n=22) and Mc3r^{+/+} (n=26) mice were given 24 hour access to two bottles, one containing plain water and the other containing ethanol in water. The concentration of ethanol (v/v) was increased every 8 days; mice received 3%, 6%, 10% and finally 20% ethanol over the course of the experiment. The positions of the bottles were changed every 2 days to control for position preferences. Average ethanol consumption/day was obtained for each ethanol concentration. To obtain a measure of ethanol consumption that corrected for individual differences in mouse size, grams of ethanol consumed/kg body weight/day were calculated for each mouse. As a measure of relative ethanol preference, an ethanol preference ratio was calculated by dividing total ethanol solution consumed by total fluid (ethanol plus water) consumption. Two-way, 2×4 (genotype×concentration) repeated measures analyses of variances (ANOVAs) were used for statistical examination of the data. All data in this report are presented as mean+S. E. M, and in all cases significance was accepted at P<0.05 (two-tailed).

Results

Relative to wild-type mice, Mc3r^{-/-} mice did not show differences in voluntary consumption of solutions containing ethanol (FIGS. 1(a) and (b)). ANOVA run on data expressed as g ethanol consumed/kg revealed a significant effect of ethanol concentration [F(3, 138)=129.94], which reflected the increased consumption of ethanol as the concentrations increased. Mice consumed approximately 5 g/kg/day of ethanol during access to the 3% solution. Consumption reached maximal levels of about 15 to 18 g/kg/day during access to 10% and 20% solutions. ANOVA run on the ethanol preference ratio data showed a significant effect of ethanol concentration [F(3, 138)=131.49]; as the concentration of ethanol increased from 10% to 20%, the ethanol preference ratio decreased below 0.5, indicating that the mice preferred water to the ethanol solution.

Mc3r^{-/-} mice drink normal amounts of solutions containing alcohol when compared to control mice (see FIGS. 1(a) and 1(b)).

### EXAMPLE 4

Ethanol Consumption Following Central Infusion of MC4R Agonist (Compound A)

Materials and Methods

The generation of Mc3r^{-/-} mice has been described (Chen, S. et al., Nature Genetics (2000) 26: 97-102). Mc3r^{-/-} mice are born at the expected frequency and are viable and fertile. Furthermore, gross anatomical and histological assessment of these mice revealed no abnormalities of brain or other organs; the Mc3r^{-/-} mice have increased fat mass, decreased lean mass, and are hypophagic. Studies described below utilized male and female Mc3r^{-/-} and/or Mc3r^{+/+} mice of a C57BL/6J genetic background, 8 to 12 weeks of age at the start of experiment. Non-littermate heterozygous (Mc3r^{+/-}) mice were bred, resulting in Mc3r^{-/-} and Mc3r ^{+/+} F2 littermate mice. Mice were individually housed in polypropylene cages with wood-chip bedding and had ad libitum access to standard rodent chow (Teklad, Madison, Wis.) and water throughout the experiments except where noted. The colony room was maintained at approximately 22° C. with a 12h:12h light:dark cycle. All procedures used in the present research were in compliance with the National Institute of Health guidelines, and the protocols were approved by the University of North Carolina Animal Care and Use Committee.

Naïve mice were anesthetized with a cocktail of ketamine (117 mg/kg) and xylazine (7.92 mg/kg) and surgically implanted with a 26-gauge cannula (Plastic One, Roanoke, Va.) aimed at the left-lateral ventricle using the following stereotaxic coordinates: 0.2 mm posterior to bregma, 1.0 mm lateral to the midline, and 2.3 mm ventral to the skull surface. Mice were allowed to recover for approximately 2 weeks before experimental procedures were initiated. After experimental procedures, cannula placement was verified histologically. The i.c.v. infusions were given in a 1.0 μl volume with a 5.0 μl Hamilton syringe and infused manually over a one minute period. Mice were acclimated for approximately 2 weeks to drinking from two bottles (continuous 24 hour/day access), one containing water and the other containing 20% ethanol, and body weights were recorded weekly. Following acclimation, mice were distributed to groups based on the last 4-days of baseline ethanol consumption (g/kg/day).

Mice were weighed and ethanol, water, and food were removed from their cages two hours before the beginning of the dark cycle. For the study with i.c.v. infusion of MC4R agonist, C57B1/6J mice were given infusion of either 1.0 or 3.0 μg of the MC4R-selective agonist, cyclo(NH—CH₂—CH₂—CO-His-D-Phe-Arg-Trp-Glu)-NH₂ (Compound A, Phoenix Pharmaceuticals, Inc., Belmont, Calif.) dissolved in aCSF (ns=7 and 8, respectively) or an equal volume of aCSF (n=10) one hour before the beginning of the dark cycle. The 20% ethanol solution, water, and food were returned immediately before the dark cycle and consumption measures were recorded 4 hours and 24 hours later. One-way (drug) ANOVAs were used to assess consumption of 20% ethanol following i.c.v. infusion of MC4R agonist or aCSF.

Results

When given centrally, the selective melanocortin 4 receptor agonist, MC4R-selective agonist, cyclo(NH—CH₂—CH₂—CO-His-D-Phe-Arg-Trp-Glu)-NH₂ (Compound A), dose-dependently lead to a 67% and 86% (1.0 and 3.0 μg, respectively) reduction of alcohol consumption [F(2, 22)=10.15] (see FIG. 2(a)).

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method of inhibiting alcohol consumption comprising administering a therapeutically effective amount of a selective melanocortin 4 receptor agonist to a subject in need thereof wherein the selective melanocortin 4 receptor agonist is a compound of Formula I: 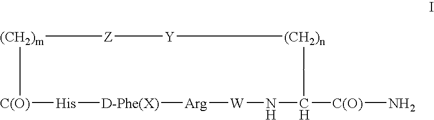
wherein:
His is L-histidyl;
D-Phe(X) is D-phenylalanyl unsubstituted or optionally para-substituted with a group selected from F, Cl, Br, Me, and OMe;
Arg is L-arginyl;
W is L-tryptophanyl or 2-naphthyl-L-alanyl;
one of Y and Z is —C(O)— and the other is —NH—;
m is 1 to 4;
n is 1 to 4, provided that n+m is 4 to 6; or
a pharmaceutically acceptable salt thereof.

2. The method of claim 1 wherein Y is —C(O)— and Z is —NH—.

3. The method of claim 2 wherein m is 2 and n is 2.

4. The method of claim 3 selected from:

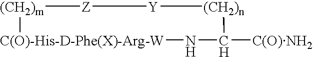

| Z | Y | X | W | m | n |
|---|---|---|---|---|---|
| NH | C(O) | H | Trp | 4 | 2 |
| NH | C(O) | H | Trp | 3 | 2 |
| NH | C(O) | H | Trp | 2 | 2 |
| NH | C(O) | H | Trp | 1 | 2 |

or a pharmaceutically acceptable salt thereof.
or a pharmaceutically acceptable salt thereof.

5. The method of claim 4 selected from cyclo(NH—CH₂—CH₂—CO-His-D-Phe-Arg-Trp-Glu)-NH₂, or a pharmaceutically acceptable salt thereof.

6. A method of reducing alcohol consumption comprising administering a selective melanocortin 4 receptor agonist, or a pharmaceutically acceptable salt thereof, to a subject in need thereof wherein the selective melanocortin 4 receptor agonist is a compound of Formula I: 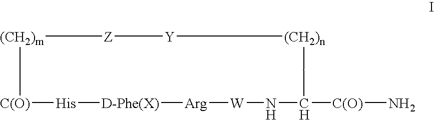
wherein:
His is L-histidyl;
D-Phe(X) is D-phenylalanyl unsubstituted or optionally para-substituted with a group selected from F, Cl, Br, Me, and Ome;
Arg is L-arginyl;
W is L-tryptophanyl or 2-naphthyl-L-alanyl;
one of Y and Z is —C(O)— and the other is —NH—;
mis 1 to 4;
n is 1 to 4, provided that n+m is 4 to 6; or
a pharmaceutically acceptable salt thereof.

7. The method of claim 6 wherein the compound of Formula I is selected from:
or a pharmaceutically acceptable salt thereof.

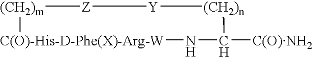

| Z | Y | X | W | m | n |
|---|---|---|---|---|---|
| NH | C(O) | H | Trp | 4 | 2 |
| NH | C(O) | H | Trp | 3 | 2 |
| NH | C(O) | H | Trp | 2 | 2 |
| NH | C(O) | H | Trp | 1 | 2 |

or a pharmaceutically acceptable salt thereof.

8. The method of claim 7 wherein the compound of Formula I is selected from cyclo(NH—CH₂—CH₂—CO-His-D-Phe-Arg-Trp-Glu)-NH₂, or a pharmaceutically acceptable salt thereof.

9. A method of treating alcoholism comprising administering a selective melanocortin 4 receptor agonist, or a pharmaceutically acceptable salt thereof, to a subject in need thereof wherein the selective melanocortin 4 receptor agonist is a compound of Formula I: 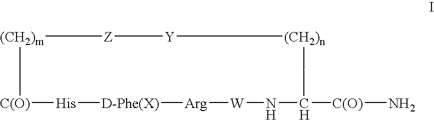
wherein:
His is L-histidyl;
D-Phe(X) is D-phenylalanyl unsubstituted or optionally para-substituted with a group selected from F, Cl, Br, Me, and OMe;
Arg is L-arginyl;
W is L-tryptophanyl or 2-naphthyl-L-alanyl;
one of Y and Z is —C(O)— and the other is —NH—;
m is 1 to 4;
n is 1 to 4, provided that n+m is 4 to 6; or
a pharmaceutically acceptable salt thereof.

10. A method of treating alcohol abuse comprising administering a selective melanocortin 4 receptor agonist, or a pharmaceutically acceptable salt thereof, to a subject in need thereof wherein the selective melanocortin 4 receptor agonist is a compound of Formula I: 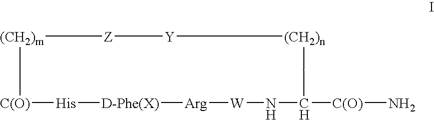
wherein:
His is L-histidyl;
D-Phe(X) is D-phenylalanyl unsubstituted or optionally para-substituted with a group selected from F, Cl, Br, Me, and OMe;
Arg is L-arginyl;
W is L-tryptophanyl or 2-naphthyl-L-alanyl;
one of Y and Z is —C(O)— and the other is —NH—;
mis 1 to 4;
n is 1 to 4, provided that n+m is 4 to 6; or
a pharmaceutically acceptable salt thereof.

11. A method of inhibiting alcohol consumption comprising administering to a subject in need thereof a therapeutically effective amount of a selective melanocortin 4 receptor agonist, or a pharmaceutically acceptable salt thereof, with a functional activity characterized by an EC₅₀ at least 15-fold more selective for the human melanocortin 4 receptor than for the human melanocortin 1 receptor, the human melanocortin 3 receptor and the human melanocortin 5 receptor.

12. The method of claim 11 wherein the functional activity of the melanocortin 4 receptor agonist is characterized by an EC₅₀ at least 17-fold more selective for the human melanocortin 4 receptor than for the human melanocortin 3 receptor.

13. The method of claim 11 wherein the functional activity of the melanocortin 4 receptor agonist is characterized by an EC₅₀ at least 90-fold more selective for the human melanocortin 4 receptor than for the human melanocortin 3 receptor.

14. The method of claim 11 wherein the functional activity of the melanocortin 4 receptor agonist is characterized by an EC₅₀ at least 200-fold more selective for the human melanocortin 4 receptor than for the human melanocortin 5 receptor.

15. The method of Claim 11 wherein the functional activity of the melanocortin 4 receptor agonist is characterized by an EC₅₀ at least 3000-fold more selective for the human melanocortin 4 receptor than for the human melanocortin 5 receptor.

16-19. (canceled)
